(19) United States
(12) Patent Application Publication
(10) Pub. No.: US 2026/0327302 A1
(43) Pub. Date: Oct. 22, 2026

(54) METHODS FOR TREATMENT OF GRAFT FAILURE

(71) Applicant: Children's Hospital Medical Center, Cincinnati, OH (US)

(72) Inventors: ANTHONY JOSEPH SABULSKI, Terrace Park, OH (US); SONATA JODELE, Hawthorne, CA (US)

(21) Appl. No.: 19/486,773

(22) PCT Filed: Jun. 7, 2024
(86) PCT No.: PCT/US2024/032938

§ 371 Date: Nov. 21, 2025

Related U.S. Application Data
(60) Provisional application No. 63/472,005, filed on Jun. 9, 2023.

(51) Int. Cl. C07K 16/24; A61K 38/19; A61K 39/395; A61P 37/06; C07K 16/18; C07K 16/28

(52) U.S. Cl. CPC C07K 16/249; A61K 38/193; A61K 39/3955; A61P 37/06; C07K 16/18; C07K 16/2866; C07K 16/2896

(57) ABSTRACT

Disclosed are methods for determining risk of transplant graft rejection of an individual, and treatment thereof. The method may comprise a) identifying a risk level of the individual as either high-risk or moderate-risk, the identification comprising determining the presence of a risk factor selected from mismatched or haploidentical donor, ex vivo T-cell depleted graft, and history of prior graft failure; and b) categorizing an individual with two identified risk factors as high-risk and an individual with one identified risk factor as moderate-risk. The method may further comprise administering an IFNy neutralizing agent to the individual identified as high-risk.

### CROSS-REFERENCE TO RELATED APPLICATIONS

This application claims priority to and benefit of U.S. Provisional Application Ser. No. 63/472,005, filed Jun. 9, 2023, the contents of which are incorporated in their entirety for all purposes.

### BACKGROUND

Graft rejection after hematopoietic stem cell transplant (HSCT) is an immune-mediated form of graft failure and leads to markedly increased mortality. Immune-mediated graft failure is a feared complication of allogeneic HSCT with no established effective intervention. Graft failure after allogeneic HSCT leads to inferior overall survival (OS) compared to engrafting patients and is an infrequent, but important cause of transplant-related mortality (TRM). Graft failure patients are significantly more likely to die within the first 6 months of their initial transplant and have a 6-month survival of <40% compared to engrafted HSCT recipient survival of >80%. (Chen J, Pang A, Zhao Y, et al. Primary graft failure following allogeneic hematopoietic stem cell transplantation: risk factors, treatment and outcomes. Hematology. December 2022;27(1):293-299. doi: 10.1080/16078454.2022.2042064.) The only available treatment for graft failure is re-transplantation, either from the same or an alternative donor. Unfortunately, even those who make it to a second transplant still have poor outcomes. A prior study of re-transplantation after primary graft failure in 122 patients showed 30-day, 100-day and 1-year survival rates of just 61%, 25% and 11%. (Chen J, Pang A, Zhao Y, et al. Primary graft failure following allogeneic hematopoietic stem cell transplantation: risk factors, treatment and outcomes. Hematology. December 2022;27(1):293-299. doi: 10.1080/16078454.2022.2042064.) TRM after re-transplantation can be as high as 70% for high-risk patients, this indicating that novel strategies aimed at prevention and/or preemptive treatment of this complication are desirable.

The lack of treatment options for graft failure is compounded by the increase in transplants likely to lead to such failure. Recent advances in graft versus host disease (GvHD) prophylaxis and transplant techniques have increased the number of mismatched and haploidentical transplants performed. Haploidentical transplants have increased nearly 5-fold over the last decade (24% of transplants in 2020 vs 5% in 2010). Several publications have characterized the incidence and pre-transplant risk factors for graft failure and donor human leukocyte antigen (HLA) mismatch as a leading risk factor. (See, e.g., Olsson et al. Graft failure in the modern era of allogeneic hematopoietic SCT. Bone Marrow Transplant. April 2013;48(4):537-43. doi: 10.1038/bmt.2012.239; Olsson et al. Primary graft failure after myeloablative allogeneic hematopoietic cell transplantation for hematologic malignancies. Leukemia. August 2015;29(8):1754-62. doi: 10.1038/leu.2015.75; Woodard P, et al. Etiology and outcome of graft failure in pediatric hematopoietic stem cell transplant recipients. J Pediatr Hematol Oncol. December 2003;25(12):955-9. doi: 10.1097/00043426-200312000-00010, and Mattsson J, Ringdén O, Storb R. Graft failure after allogeneic hematopoietic cell transplantation. Biol Blood Marrow Transplant. January 2008;14(1 Suppl 1):165-70. doi: 10.1016/j.bbmt.2007.10.025.) Graft rejection rates are therefore expected to increase in the setting of increasing mismatched donor transplants, increasing the demand for improved prophylaxis and/or treatment for this complication.

As noted above, there are no reliable graft rejection interventions outside of re-transplantation, and no accepted therapeutic intervention to prevent an impending rejection. There is similarly no consensus in the HSCT community regarding the appropriate approach to these patients. The ASTCT recently published the results of an expert panel of HSCT physician recommendations for the management of graft failure after HSCT. (Kharfan-Dabaja MA, Kumar A, Ayala E, et al. Standardizing Definitions of Hematopoietic Recovery, Graft Rejection, Graft Failure, Poor Graft Function, and Donor Chimerism in Allogeneic Hematopoietic Cell Transplantation: A Report on Behalf of the American Society for Transplantation and Cellular Therapy. Transplant Cell Ther. August 2021;27(8):642-649. doi: 10.1016/j.jtct.2021.04.007.) No consensus was reached and conflicting recommendations of “increase immune suppression” and “decrease immune suppression” were recommended by different panel members. Additionally, graft rejection was identified as the most expensive complication of HSCT in a multivariate analysis of 202 HSCT recipients (p<0.001). (Svahn BM, et al., Increased costs after allogeneic haematopoietic SCT are associated with major complications and re-transplantation. Bone Marrow Transplant. May 2012;47(5):706-15. doi: 10.1038/bmt.2011.162.) Re-transplantation was found to double the 1-year cost of HSCT in the same study (p<0.001) Id. Thus, there is an urgent need for effective interventions for one or both of graft failure prevention or improved graft failure treatment. The instant disclosure seeks to address one or more of the aforementioned needs in the art.

### BRIEF SUMMARY

Disclosed are methods for determining risk of transplant graft rejection of an individual, and treatment thereof, particularly for those patients receiving HSCT. The method may comprise a) identifying a risk level of the individual as either high-risk or moderate-risk, the identification comprising determining the presence of a risk factor selected from mismatched or haploidentical donor, ex vivo T-cell depleted graft, and history of prior graft failure; and b) categorizing an individual with two identified risk factors as high-risk and an individual with one identified risk factor as moderate-risk. The method may further comprise administering an IFNγ neutralizing agent to the individual identified as high-risk.

### BRIEF DESCRIPTION OF THE DRAWINGS

This application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 depicts a graph showing absolute neutrophil count kinetics following emapalumab therapy for suspected graft failure.

FIG. 2 depicts a bar chart showing time to absolute neutrophil count recovery in responders following emapalumab administration.

FIG. 3 depicts graphs showing CXCL9 elevations in subjects who developed primary and secondary graft failure.

FIG. 4 depicts a graph of CXCL9 levels in patients who received prophylactic emapalumab to prevent graft failure (GF).

FIG. 5 depicts Table 1, Patient demographics and outcomes following preemptive “treatment” emapalumab to prevent graft failure.

FIG. 6 depicts Table 2, Demographics and transplant data from prophylactic emapalumab cohort.

FIG. 7 depicts Table 3, CTC-AE grading of adverse events after emapalumab therapy in patients treated peri-transplant for graft failure.

FIG. 8 depicts a flow chart of a Pre-Transplant Rejection Risk Assessment and Intervention for identifying and treating High Risk and Moderate Risk patients.

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein may be used in practice or testing of the present invention. All publications, patent applications, patents and other references mentioned herein are incorporated by reference in their entirety. The materials, methods, and examples disclosed herein are illustrative only and not intended to be limiting. The methods may comprise, consist of, or consist essentially of the elements of the methods as described herein, as well as any additional or optional element described herein or otherwise useful in treating graft failure, in particular graft failure following an HSCT protocol.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” may mean within 1 or more than 1 standard deviation, per the practice in the art. Alternatively, “about” may mean a range of up to 20%, or up to 10%, or up to 5%, or up to 1% of a given value. Alternatively, particularly with respect to biological systems or processes, the term may mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “effective amount” means the amount of one or more active components that is sufficient to show a desired effect. This includes both therapeutic and prophylactic effects. When applied to an individual active ingredient, administered alone, the term refers to that ingredient alone. When applied to a combination, the term refers to combined amounts of the active ingredients that result in the therapeutic effect, whether administered in combination, serially or simultaneously.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

As used herein, the term “treat”, “treating”, or “treatment” means managing a patient's condition, and includes the administration of a substance to prevent the onset of the symptoms or complications, alleviating the symptoms or complications, or eliminating the disease or condition. This could be achieved by halting, slowing, or reversing the progression of the disease or condition, causing the regression of the disease or condition, or preventing the disease or condition from recurring. This could also include palliative treatment, where the symptoms of the disease or condition are alleviated, but the disease or condition is not cured.

As used herein, the term “prophylaxis” or “prophylactic treatment” refers to measures taken to prevent the onset of a particular disease or health condition. This may include the administration of an active agent or other therapeutic intervention designed to confer resistance against a specific disease or condition.

Disclosed are methods for treating an individual having, or at risk of having graft rejection. In aspects, the individual having, or at risk of having graft rejection is one who has undergone hematopoietic stem cell transplantation (HSCT). The disclosed methods may be particularly suited for prophylactic identification of an individual likely to develop graft failure, and optionally, prophylactic treatment of graft failure in an individual having undergone HSCT, wherein the presence or absence of a risk factor is determined, and wherein a IFNγ neutralizing agent such as emapalumab is administered to the individual when at least two risk factors as disclosed herein are present. In other aspects, the disclosed methods may be used solely as a prophylactic method to diagnose and optionally, treat an individual. In other aspects, the disclosed methods may be used both prophylactically and remedially to diagnose and, optionally, treat an individual for graft failure. In other aspects, the disclosed methods may be used to remedially diagnose and, optionally, treat an individual for graft failure. In aspects, the remedial treatment may comprise treating early stage graft failure before a full graft failure has taken place.

In one aspect, method comprises
identifying a risk level of the individual as either high-risk or moderate-risk, the identification comprising determining the presence of a risk factor selected from mismatched or haploidentical donor, ex vivo T-cell depleted graft, and history of prior graft failure; and
categorizing an individual with at least two identified risk factors as high-risk for graft rejection and an individual with one identified risk factor as moderate-risk for graft rejection.

In aspects, the disclosed methods comprise characterizing an individual as being high-risk for graft rejection or moderate-risk for graft rejection. In this aspect, the determination is based on assessment of the following risk factors: the use of a mismatched or haploidentical donor, the use of an ex vivo T-cell depleted graft, and the presence of donor specific antibodies. Determination of the risk factors is within the skill of one of ordinary skill in the art. For example, the risk factors, and determination thereof for purposes of carrying out the disclosed invention are provided as follows.

### Mismatched or Haploidentical Donor Risk Factor

A first risk-factor may comprise a patient having a mismatched donor. Human Leukocyte Antigen (HLA) genes may be used to determine donor compatibility. Matching HLA genes between donor and recipient may significantly improve the success of the transplant and reduce the risk of complications such as graft-versus-host disease (GVHD). 10 loci are typically considered for HLA matching: HLA-A, HLA-B, HLA-C, HLA-DRB1, HLA-DQB1, and HLA-DPB1 (HLA-DPB1 being on two different chromosomes). In aspects, a mismatched donor is a donor that has less than a “ 10/10 match,” i.e., with at least one recognized HLA allele mismatch (i.e., 9/10 or less). A 10/10 match, for purposes of the instant disclosure, means that all 10 alleles are identical between the donor and recipient. Determination of HLA alleles may be performed using methods well known in the art, such determination typically including sample collection (blood sample or buccal swab) and subsequent DNA sequencing to determine the HLA alleles. Exemplary methods of HLA typing are described in Sakaue, S., Gurajala, S., Curtis, M. et al. Tutorial: a statistical genetics guide to identifying HLA alleles driving complex disease. Nat Protoc 18, 2625-2641 (2023). doi.org/10.1038/s41596-023-00853-4.

The first risk factor may comprise a patient having a haploidentical donor. Haploidentical transplants have become increasingly common due to improvements in transplant techniques and the fact that nearly every patient will have at least one haploidentical donor within their family, typically a parent or sibling. However, these transplants can be associated with a higher risk of complications, such as graft-versus-host disease (GVHD) and graft failure, compared to transplants from fully matched donors. A haploidentical donor is a type of donor for hematopoietic stem cell transplantation (HSCT) in which the donor and recipient share one identical set of human leukocyte antigen (HLA) genes, or one parental haplotype, also referred to as a “half match”. As would be readily understood by one of ordinary skill in the art, an individual has two sets of HLA genes, one from each parent, known as haplotypes; in a haploidentical transplant, the donor and recipient share one of these haplotypes, hence the term “haploidentical” (haplo- means half). The remaining HLA alleles (the other haplotype) are not identical between the donor and recipient and are divided randomly. This means that the degree of mismatch in the other haplotype can vary. Therefore, a haploidentical match is not restricted to a 5/10 match (where 5 out of 10 considered HLA alleles match), but may include a 6/10 match, or even higher, depending on the specific alleles present in the non-shared haplotype.

### Ex Vivo T-Cell Depleted Graft

The second risk factor may be the individual receiving an ex vivo T-cell depleted graft. As used herein, an ex vivo T-cell Depleted Graft refers to a graft wherein T-cells have been removed ex vivo, or outside the body, which is commonly used in HSCT. To mitigate the risk of Graft-versus-Host Disease (GvHD), a major complication post-transplant, T-cells can be depleted from the graft ex vivo prior to infusion. In one aspect, the ex vivo T-cell depletion is obtained via positive selection. In one aspect, the ex vivo T-cell depletion is obtained via negative depletion. In positive selection desired cells (typically CD34+ hematopoietic stem cells) are retained while discarding the remaining cells, which may be achieved, for example, via immunomagnetic separation. In negative depletion, targeted removal of undesired cells (e.g., T-cells), may be employed, leaving behind the desired cells. Negative depletion may be achieved using immunomagnetic separation in which T-cells are labeled and removed. Methods of ex vivo T-Cell Depleted Graft, and identification of individuals who are recipients of same is well understood by one of ordinary skill in the art.

### History of Prior Graft Failure

In one aspect, the risk factor is a history of prior graft failure. Graft failure is a serious complication that can occur after a hematopoietic stem cell transplantation (HSCT), defined as the failure to achieve sustained engraftment following the transplantation. Graft failure may be divided into primary and secondary failure. Primary Graft Failure is characterized by the absence of any hematological function of the graft and is defined by failure to achieve a neutrophil count >0.5×10⁹/L within 28 days of stem cell infusion. Secondary Graft Failure occurs after evidence of donor engraftment. After initial evidence of neutrophil recovery, the count falls below 0.5×10⁹/L. This is almost always accompanied by significant thrombocytopenia (platelets<30×10⁹/L) and anemia. The clinical criteria for graft failure include a lack of sustained engraftment as defined above, and usually, in graft failure, the neutrophil count will be <0.1×10\hat{ }9/L1. (See, e.g., Rostami, T., Rostami, M.R., Mirhosseini, A.H. et al. Graft failure after allogeneic hematopoietic stem cell transplantation in pediatric patients with acute leukemia: autologous reconstitution or second transplant?. Stem Cell Res Ther 15, 111 (2024). doi.org/10.1186/s13287-024-03726-z.) In one aspect, the risk factor is primary graft failure. In one aspect, the risk factor is secondary graft failure.

### Application of Risk Factors

Based on an assessment of donor status, T-Cell graft status, and history of prior graft failure, as described above, the individual is categorized as high-risk status, moderate risk status, or neither. In aspects, the individual may have two risk factors, and is categorized as high-risk for graft rejection (“high-risk patient”). In aspects, the individual may have all three risk factors, and is categorized as high-risk for graft rejection. In aspects, the individual has only one risk factor, and is categorized as moderate-risk for graft rejection. An individual having one of the aforementioned risk factor is characterized as being at moderate risk for graft failure (“moderate risk patient”). A patient not identified as having neither high-risk status nor moderate-risk status is not characterized as either high risk or moderate risk, and will remain under normal care with daily monitoring of symptoms.

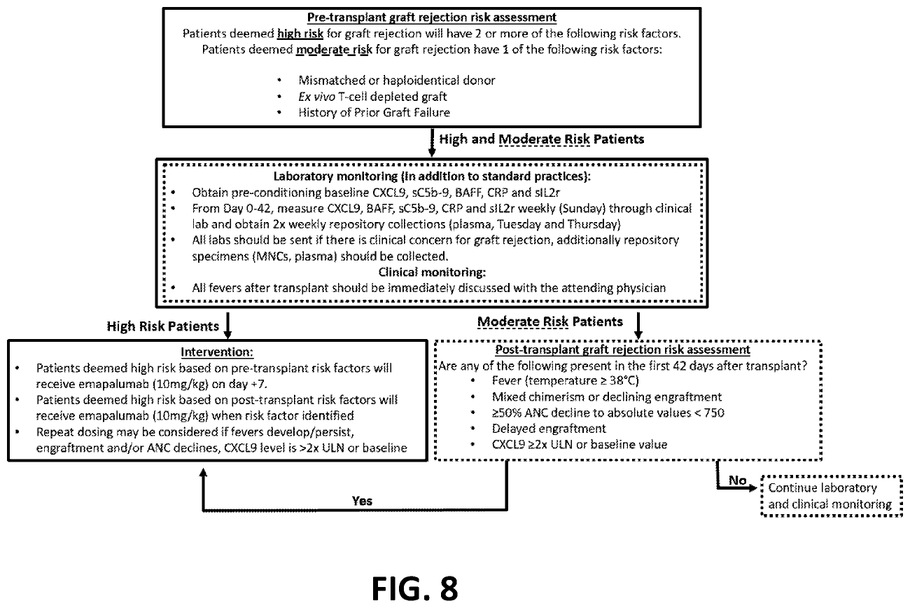

Referring to FIG. 8, for both High-Risk Patients and Moderate-Risk Patients, laboratory monitoring and clinical monitoring is carried out. Laboratory monitoring includes: 1) obtaining pre-conditioning baseline CXCL9, sC5b-0, B Cell Activating Factor Level (BAFF), CRP, and sIL2r; and 2) measuring CXCL9, BAFF, sC5b-9, CRP, and sIL2r weekly, from day 0-42 twice a week. CXCL, BAFF, and sC5b-9 have been described as potential biomarkers for graft rejection after transplant. See, e.g., Sabulski, Anthony et al. “Graft rejection markers in children undergoing hematopoietic cell transplant for bone marrow failure.” Blood advances vol. 5,22 (2021): 4594-4604. doi: 10.1182/bloodadvances.2021005231. Thus, in aspects, the method further comprises detecting a pre-conditioning baseline level of a biomarker selected from CXCL9, sC5b-9, BAFF, CRP, sIL2r, and combinations thereof in the individual. Further referring to FIG. 8, clinical monitoring is carried out for both high-risk and moderate-risk patients. In aspects, the clinical monitoring comprises monitoring the patient for fever after transplant.

A detailed description of the biomarkers assayed during pre-conditioning are as follows:
CXCL9 (Chemokine (C-X-C motif) ligand 9) is a small cytokine that belongs to the CXC chemokine family. The detection and quantification of the CXCL9 can be carried out using methods well known in the art. For example, an enzyme-linked immunosorbent assay (ELISA) can be used to detect and quantify the biomarker in a sample. Those skilled in the art will appreciate that other types of assays can also be used to determine CXCL9 levels. In one aspect, quantification of CXCL9 serum levels may be determined using Validated MesoScale Discovery (MSD, Rockville, MD, USA) platform-based immunoassay. CXCL9 levels may be measured in the blood or plasma of an individual and normalized to a control value. In aspects, the value is normalized to the upper limit of normal (ULN).
sC5b-9. sC5b-9, also known as the Soluble Terminal Complement Complex, is a product of the activation of the complement system, which is a part of the immune system. The detection and quantification of the sC5b-9 can be carried out using methods well known in the art. For example, an enzyme-linked immunosorbent assay (ELISA) can be used to detect and quantify the biomarker in a sample. Those skilled in the art will appreciate that other types of assays can also be used to determine sC5b-9 levels.
B Cell Activating Factor Level (BAFF), also known as tumor necrosis factor ligand superfamily member 13B and CD257 among other names, is a protein that in humans is encoded by the TNFSF13B genel. BAFF is a cytokine that belongs to the tumor necrosis factor (TNF) ligand family. The detection and quantification of BAFF can be carried out using methods well known in the art. For example, an enzyme-linked immunosorbent assay (ELISA) can be used to detect and quantify the biomarker in a sample. Those skilled in the art will appreciate that other types of assays can also be used to determine BAFF levels.
C-reactive protein (CRP) is a protein produced by the liver that increases in the blood when there's inflammation in the body. The detection and quantification of CRP can be carried out using methods well known in the art. For example, an enzyme-linked immunosorbent assay (ELISA) can be used to detect and quantify the biomarker in a sample. Those skilled in the art will appreciate that other types of assays can also be used to determine CRP levels.
Soluble interleukin-2 receptor (sIL-2R), also known as soluble CD25, SIL-2R, HLH: soluble IL-2R, interleukin 2R, Soluble IL2R, sCD25, and SCD25, may be detected and quantified using methods well known in the art. For example, an enzyme-linked immunosorbent assay (ELISA) can be used to detect and quantify the biomarker in a sample. Those skilled in the art will appreciate that other types of assays can also be used to determine sIL-2R levels.
ANCs, or Absolute Neutrophil Counts, are a measure of the number of a specific type of white blood cells called neutrophils in the blood. Determination of ANC is well known and is typically determined via a standard Complete Blood Count (CBC) study.

Further referring to FIG. 8, following characterization of an individual as either moderate-risk for graft rejection (moderate-risk status) or high-risk for graft rejection (high-risk status), an intervention (for high-risk patients) or post-transplant graft rejection risk assessment protocol (for moderate-risk patients) is carried out.

For moderate-risk patients, the individual is subjected to post-transplant graft rejection risk assessment. The post-transplant graft rejection risk assessment comprises monitoring for at least one of the following: fever (temperature equal to or greater than 38 °C, mixed chimerism or declining engraftment, greater than or equal to ANC decline to absolute values less than 750 (ANC drops by 50% or greater from a previous value and the number it drops to is less than 0.75×10³ cells/microliter (equivalent to 750 cells/microliter)), delayed engraftment, CXCL9 equal to or greater than 2×ULN or baseline value. If none of the post-transplant graft rejection risk factors are present, laboratory and clinical monitoring are continued. If at least one of the post-transplant graft rejection risk factors are present, the individual is reclassified as being high-risk for graft rejection (high-risk status). In aspects, the individual is identified as having fever, engraftment and/or ANC decline, CXCL9 level of greater than 2× upper limit of normal (ULN) or baseline and is administered an IFNγ neutralizing agent. In further aspects, the method may comprise detecting in an individual classified as moderate-risk, the presence of a biomarker selected from fever, mixed chimerism, declining engraftment, an ANC decline of greater than or equal to 50% to absolute values less than 750, delayed engraftment, or CXCL9 greater than or equal to two times ULN or baseline value; and administering the IFNγ neutralizing agent (e.g., emapalumab) to the individual when the detected biomarker has a level that is elevated compared to a baseline level. In aspects, the baseline level may be a level detected in the individual prior to transplant. In other aspects, the baseline level may be a control value that is representative of a healthy individual, which may further be age and/or sex matched.

Referring again to FIG. 8, following identification of an individual as being at high-risk for graft rejection (high-risk patient), an intervention is applied to the high-risk patient. In aspects, where the patient is determined to be a high-risk patient based on pre-transplant risk factors, the intervention is administration of an IFNγ neutralizing agent. In aspects, the IFNγ neutralizing agent is a human anti-IFNγ antibody. In aspects, the IFNγ neutralizing agent is emapalumab. Emapalumab (GAMIFANT, Novimmune SA) is a monoclonal antibody that binds to and neutralizes interferon gamma (IFNγ), blocking its intracellular signaling to inhibit macrophage activation and the downstream release of proinflammatory cytokines. Emapalumab is approved for hemaphagocytic lymphohistiocytosis. www.fda.gov/drugs/fda-approves-emapalumab-hemophagocytic-lymphohistiocytosis. Emapalumab has shown promising efficacy in the treatment of patients with graft failure (GF) requiring a second allogeneic hematopoietic stem cell transplantation (HSCT). Tucci F, Gallo V, Barzaghi F, et al. Emapalumab treatment in an ADA-SCID patient with refractory hemophagocytic lymphohistiocytosis-related graft failure and disseminated bacillus Calmette-Guérin infection. Haematologica. 2021;106(2):641-646. Published 2021 Feb. 1. doi: 10.3324/haematol.2020.255620. Emapalumab is described in, for example, WO 2006/109191, WO 2016/177913 and WO 2018/078442.

In aspects, the patient is determined to be a high-risk patient based on pre-transplant risk factors and emapalumab is administered on “day+7” (7 days after transplant, wherein day 0 is stem cell infusion day). Where the patient is determined to have a high-risk status (a “high-risk patient”) based on post-transplant risk factors, the intervention is administration of emapalumab. In aspects, the patient is determined to have a high-risk status based on post-transplant risk factors and emapalumab is administered when the risk factor is identified.

In aspects, the individual is characterized as having a high-risk status, based on the risk factors set forth above, and is administered an IFNγ neutralizing agent (e.g., a human anti-IFNγ antibody, e.g., emapalumab). In aspects, the IFNγ neutralizing agent is emapalumab and is administered at a dosage of at least 5 mg/kg, or at least 6 mg/kg, or at least 7 mg/kg, or at least 8 mg/kg, or at least 9 mg/kg, or at least 10 mg/kg. In other aspects, the IFNγ neutralizing agent is emapalumab and is administered in amounts of about 10 mg/kg to about 20 mg/kg, or about 10 mg/kg to about 15 mg/kg, or about 7 to about 12 mg/kg. In aspects, the dosage is administered as a single dose over one hour, via IV administration. In aspects, the administering comprises administering about 10 mg/kg of emapalumab to the individual. In aspects, the administering comprises administering about 10 mg/kg of emapalumab to the individual at day 7 post-transplant. In aspects, the administering comprises administering about 10 mg/kg of emapalumab to the individual at the time when the individual is categorized as high-risk.

In aspects the dosing of the IFNγ neutralizing agent (e.g., a human anti-IFNγ antibody, e.g., emapalumab) is repeated. In one aspect, a repeat dosing may be administered if a fever develops.

In one aspect, a repeat dosing may be administered if a fever persists, e.g., if fever lasts longer than one day. In one aspect, a repeat dosing may be administered if a fever persists, e.g., if fever lasts longer than two days. In one aspect, a repeat dosing may be administered if a fever persists, e.g., if fever lasts longer than three days. In one aspect, a repeat dosing may be administered if a fever persists, e.g., if fever lasts longer than four days. In one aspect, a repeat dosing may be administered if a fever persists, e.g., if fever lasts longer than five days. In one aspect, a repeat dosing may be administered if a fever persists, e.g., if fever lasts longer than six days. In one aspect, a repeat dosing may be administered if a fever persists, e.g., if fever lasts longer than a week. In one aspect, a repeat dosing may be administered if a engraftment declines. In one aspect, a repeat dosing may be administered if ANC declines. In one aspect, a repeat dosing may be administered if CXCL9 is greater than 2×ULN. For example, the level of CXCL9 may be measured weekly, wherein a value that is more than double the upper limit of normal indicates that an additional dose should be administered. In aspects, the level of CXCL9 may be measured at least twice a week or twice a week, wherein, wherein a value that is more than double the upper limit of normal indicates that an additional dose should be administered. The amount and duration of dosing may be the same as that for the first dose of emapalumab. That is, the second dose may be at least 5 mg/kg, or at least 6 mg/kg, or at least 7 mg/kg, or at least 8 mg/kg, or at least 9 mg/kg, or at least 10 mg/kg. In other aspects, the second dose of emapalumab may be administered in amounts of about 10 mg/kg to about 20 mg/kg, or about 10 mg/kg to about 15 mg/kg, or about 7 to about 12 mg/kg. In aspects, the second dose is administered as a single dose over one hour, via IV administration. In aspects, the second dose is about 10 mg/kg of emapalumab.

In one aspect, disclosed is a method for treating graft failure after allogeneic hematopoietic stem cell transplant (HSCT) in an individual in need thereof, the method comprising administering 1 to 10 mg/kg, as a 1-hour intravenous infusion of a human anti-IFNγ antibody, for example, emapalumab, to the individual. The individual may be an individual having high-risk status. Alternatively, the individual may be an individual having moderate-risk status. In further aspects, the individual may be identified as being neither high nor moderate risk but may be administered an ant-IFNγ antibody prophylactically. In aspects, the treating may be a prophylactic treatment comprising administration of emapalumab. In further aspects, the method may comprise administering eculizumab to the individual, in combination with the emapalumab, wherein the eculizumab and emapalumab are administered simultaneously or sequentially. In aspects, the method further comprises administering one or both of tocilizumab and daratumumab to the individual.

### Clinical Decision Support Devices

Further disclosed are clinical support devices for carrying out the disclosed methods, or a portion thereof. In aspects, the disclosed methods may be carried out using a device comprising a computer for inputting one or more of the data points obtained above, for example, a risk status and/or a biomarker status or value as described above. The device may be used for computation of a predictive value, e.g., a risk status, to produce an output for presentation to a clinician. In aspects, the device may be used for computation of an enrollment recommendation for enrollment of the subject into a clinical trial. In further aspects, disclosed is a device comprising a processor configured to receive data obtained according to the method set forth herein. In aspects, the device may be configured to determine a risk status in an individual, based on the data obtained using a method as disclosed herein. The device may be configured to provide a risk status to a clinician, for the administration of emapalumab. In aspects, a computer program product stored on a non-transitory computer-readable medium is disclosed, the computer program product comprising instructions that, when executed by a processor, cause the processor to determine a risk status to a clinician for the administration of emapalumab. The computer program may be used to determine the risk status according to the method disclosed herein.

The following non-limiting examples are provided to further illustrate embodiments of the invention disclosed herein. It should be appreciated by those of skill in the art that the techniques disclosed in the examples that follow represent approaches that have been found to function well in the practice of the invention, and thus may be considered to constitute examples of modes for its practice. However, those of skill in the art should, in light of the present disclosure, appreciate that many changes may be made in the specific embodiments that are disclosed and still obtain a like or similar result without departing from the spirit and scope of the invention.

### Example 1

Immune-mediated graft failure is a feared complication of allogeneic hematopoietic stem cell transplant (HSCT) and there is currently no established effective intervention. Multiple pre-transplant risk factors for graft failure are known and recent studies have identified interferon gamma (IFNγ) as a mechanistically important and pharmacologically targetable cytokine involved in graft failure pathophysiology. CXCL9, a downstream marker of IFNγ production, has also been identified as a biomarker. Applicant carried out a multicenter retrospective pooled analysis of emapalumab, an IFNγ neutralizing agent, for the prevention/treatment of graft failure after HSCT. Two different strategies were carried out: 1) treatment and 2) prophylaxis. In the treatment cohort, emapalumab was used in 25 HSCTs wherein patients developed clinical and laboratory signs of graft failure. All patients tolerated emapalumab without complications and no side effects or infections were attributed to emapalumab. Fifty six percent ( 14/25) of patients treated with emapalumab for impending graft failure engrafted and had resolution of graft failure concerns, while the remaining patients suffered graft failure. Absolute neutrophil count improvement was seen within 3-7 days in responders. In the prophylactic cohort, 11 patients received emapalumab based on a prior history of graft failure or other pre-transplant risk factors. Emapalumab was well tolerated and 91% ( 10/11) of patients engrafted while one patient with prior history of graft failure suffered recurrent graft failure. These safety and preliminary efficacy observations support a larger prospective study of IFNγ blockade for graft failure prevention after HSCT.

Graft failures are mechanistically divided into immune-mediated and non-immune-mediated events. Immune-mediated graft failure, often termed graft rejection, occurs when the residual host immune system attacks and eliminates donor hematopoietic cells. Non-immune-mediated graft failure events include those attributable to post-graft infusion toxic effects on hematopoietic stem cells (HSC) and/or inadequate stem cell dose. Successful intervention is unlikely for recipients of an inadequate cell dose outside of provision of additional stem cells, but graft failure that is immune-mediated could potentially be treated with immunomodulation. Prior studies of immune-mediated graft failure reported the essential contribution of interferon gamma (IFNγ) to graft failure pathophysiology, which makes IFNγ blockade a rational therapy for the prevention of immune-mediated GF.

Emapalumab is a human anti-IFNγ antibody that is FDA-approved for the treatment of refractory/relapsed primary hemophagocytic lymphohistiocytosis (HLH). Given the established role of IFNγ in the pathophysiology of immune-mediated graft failure, some transplant centers have used emapalumab during HSCT to prevent or treat impending graft failure. Described herein is a multicenter retrospective pooled analysis of emapalumab therapy for the prevention/treatment of graft failure after HSCT. The safety and preliminary efficacy of this therapy in patients who received this therapy is reported using two different strategies: 1) treatment and 2) prophylaxis.

### Methods

Data was collected retrospectively from six European and US transplant centers (Bambino Gesù Children's Hospital (OPBG) in Rome, Helsinki University Central Hospital in Helinski, Cincinnati Children's Hospital Medical Center (CCHMC), Cincinnati, OH, Children's Hospital of Los Angeles, Los Angeles, CA, Mayo Clinic, Rochester, MN, and Children's Hospital of Pittsburgh, Pittsburgh, PA) who treated patients with emapalumab with the aim of supporting sustained donor-cell engraftment. All patients included in the study received at least one dose of emapalumab between the beginning of conditioning and 4 months after HSCT for the purpose of preventing or treating graft failure. Emapalumab (SOBI, Stockholm, Sweden) was administered according to different schedules and doses (described below and in results), ranging from 1 to 10 mg/kg, as a 1-hour i.v. infusion after premedication as per local protocol. Adverse events were collected following the first dose of emapalumab to 30 days after the last administration of the drug and graded according to CTC-AE v5.

Patients at US transplant centers were treated with emapalumab if they developed clinical or laboratory signs of graft failure after HSCT. This approach is described as “treatment.” All treated patients met 2 or more of the following criteria: 1) HLA mismatched donor and/or prior graft failure, 2) unexplained fever >39 °C (or >38 °C if prior history of graft failure) after HSCT, 3) absolute neutrophil count decline after initial engraftment, 4) delayed neutrophil engraftment after HSCT, 5) real-time CXCL9>2.5×ULN (cutoff value chosen based on prior study of CXCL9 and graft failure). These criteria were chosen based on previously published risk factors for graft failure and prior studies that describe high fever as a common finding in GF.

Patients treated at European transplant centers received emapalumab to prevent graft failure if they were deemed at high-risk for graft failure prior to transplant. This approach is therefore described as “prophylactic.” High-risk patients either had a prior history of graft failure after HSCT or had two or more of the following risk factors for graft failure: a) affected by a disease with high risk of graft failure, including acquired severe aplastic anemia, thalassemia, primary HLH; b) ex-vivo T cell-depleted stem cell product; c) transplant from unrelated cord blood unit or mismatched related/unrelated donor.

Because CXCL9 has been proposed as a biomarker of IFNγ activity and its neutralization during anti-IFNγ therapies correlates with response to treatment, peripheral blood (PB) samples were collected at different time points after HSCT to measure this and other chemokines. Blood samples were collected at European centers on day 0, +3±2, +7±2, +10±2, +14±2, +30±2 after transplantation. Validated MesoScale Discovery (MSD, Rockville, MD, USA) platform-based immunoassay was used for the quantification of CXCL9 serum levels at OPBG. CXCL9 levels were measured clinically by the CLIA-certified Diagnostic Immunology Laboratory at CCHMC in patients treated at US transplant centers. CCHMC patients had CXCL9 levels measured weekly at minimum throughout the transplant course. Other US centers measured CXCL9 levels at the time of graft failure concern, prior to emapalumab dosing. Values were normalized per upper limit of normal (ULN) to account for differences in the sensitivity and normal values for these different platforms.

Quantitative variables were reported as median value and range, while categorical variables were expressed as absolute value and percentage. The Mann-Whitney rank sum test or the Student's T-test were used for continuous variables. Statistical analysis was performed using GraphPad version 8.0 (GraphPad Software, San Diego, USA). This retrospective pooled study was approved by the IRBs at all participating children's hospitals.

### Results

### Patient and Transplant Characteristics and Emapalumab Administration

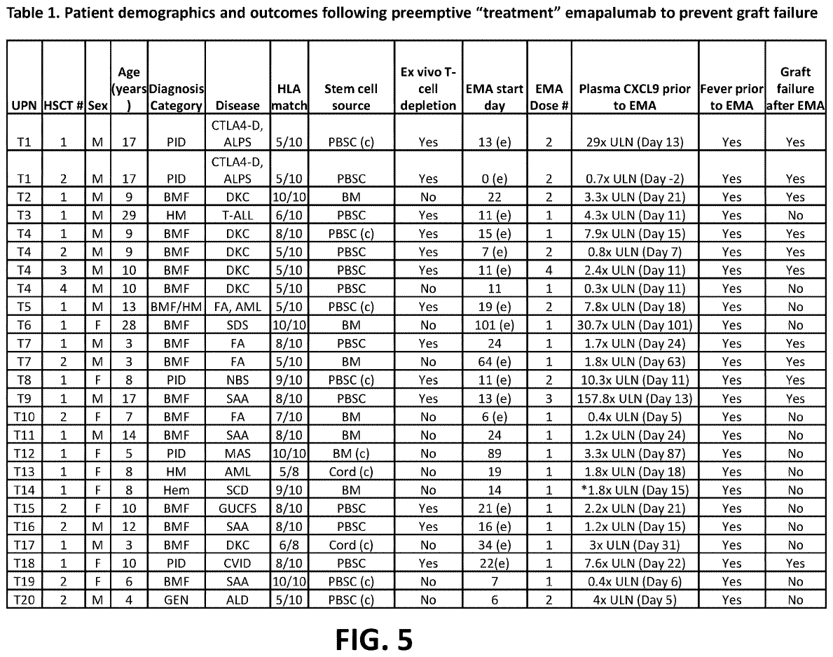

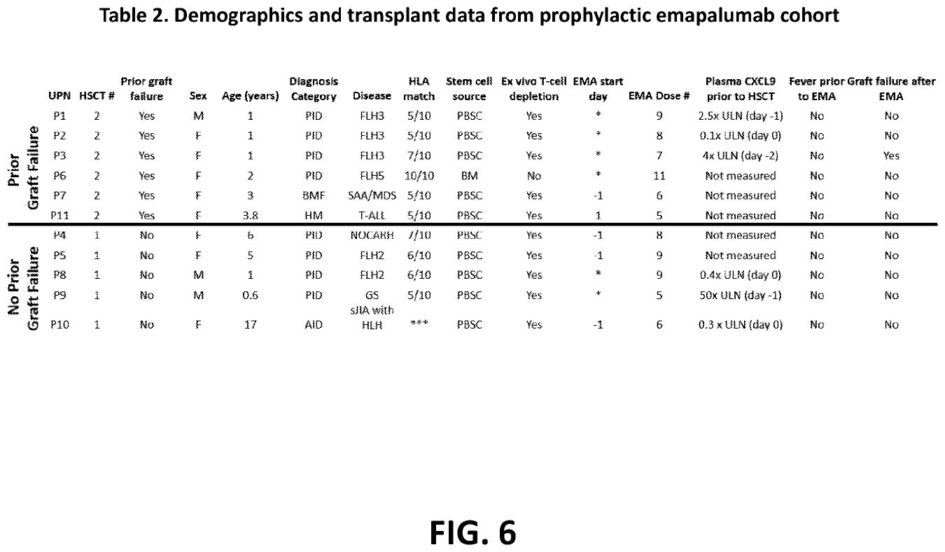

Emapalumab was administered during 36 transplants in 31 patients. Schedule and dose of emapalumab varied depending on the treating center. Patients and transplant characteristics are detailed in Table 1 and 2, according to the treatment aim: treatment (US centers) or prophylaxis (European centers). Six patients (#E1-E4 and T15-T16) have been previously reported. FIG. 5 depicts Table 1: Patient demographics and outcomes following preemptive “treatment” emapalumab to prevent graft failure. The (c) in the stem cell source column indicates the product was cryopreserved. The (e) in the EMA start day column indicates eculizumab was also given. T20 had two separate and distinct events concerning for graft failure: the first was on day 6, the second was on day 97. The value for Subject T14 (indicated by an *) is from the next day. T15 and T16 were in a prior publication (Sabulski et al. Blood Advances, 2021). Abbreviations used in Table 1 are as follows. ALD=adrenoleukodystophy, ALPS=autoimmune lymphoproliferative syndrome, AML=acute myeloid leukemia, BM=bone marrow, BMF=bone marrow failure, CTLA4-D=CTLA4 deficiency, DKC=dyskeratosis congenita, (e)=eculizumab, EMA=emapalumab, FA=Fanconi Anemia, GEN=genetic condition, GUCFS=genetically undefined chromosomal fragility syndrome, Hem=hemoglobinopathy, HLA=human leukocyte antigen, HM=hematologic malignancy, HSCT=hematopoietic stem cell transplant, MAS=macrophage activation syndrome, NBS=Nijmegen breakage syndrome, PBSC=peripheral blood stem, cell, PID=primary immune deficiency, SAA=severe aplastic anemia, SCD=sickle cell disease, SDS=Schwachman Diamond syndrome, ULN=upper limit of normal. FIG. 6 depicts Table 2: Demographics and transplant data from prophylactic emapalumab cohort. These patients indicated by an “*” received emapalumab maintenance therapy for the control of HLH leading up to HSCT and continued treatment during HSCT. For these patients dose numbers include the number of doses administered starting from day −1 since this is when prophylactic emapalumab was started in non-HLH patients. P1-4 were in prior publications (Merli et al. 2018; Lam et al, J Exp Med 2019). Abbreviations used in Table 2 are as follows. AID=autoimmune disease, BM =bone marrow, BMF=bone marrow failure, EMA=emapalumab, FLH=familial lymphohistiocytosis; GS=Griscelli syndrome; HLA=human leukocyte antigen, HM=hematologic malignancy, HSCT=hematopoietic stem cell transplant; NOCARH=neonatal onset of cytopenia, autoinflammation, rash, and episodes of hemophagocytic lymphohistiocytosis syndrome; PBSC=peripheral blood stem, cell, PID=primary immune deficiency, SAA=severe aplastic anemia, sJIA=Systemic Juvenile Idiopathic Arthritis, ULN=upper limit of normal.

Patients in the “treatment group” (n=20 patients, n=25 HSCTs) received emapalumab therapy once signs and symptoms of graft failure appeared with the aim of preventing impending graft failure. Patient demographics and transplant details are shown in Table 1. Median age at HSCT was 9 years (range, 3-29). All treated patients were febrile at treatment and mismatched donors were commonly used in these HSCTs (84%, 21/25 donors). The first dose of emapalumab was given a median of 15 days after HSCT (range, 0-101). One or two doses of emapalumab were used in 92% ( 23/25) of HSCTs. The remaining two patients (T9 and T4) received 3 and 4 doses of emapalumab, respectively. Emapalumab re-dosing was based on the persistence of graft failure signs/symptoms and real-time CXCL9 level monitoring. Three patients in this cohort had previously developed graft failure and received emapalumab for recurrent graft failure concerns in one or more subsequent HSCTs (T1, T4, T7). T4 achieved stable engraftment after his 4th HSCT while T1 and T7 suffered graft failure after their second HSCTs and did not undergo a third HSCT.

Eculizumab (a C5 inhibitor) was administered concurrently with emapalumab in 64% of the HSCTs ( 16/25) due to concurrent thrombotic microangiopathy (TMA) or based on prior evidence that suggests terminal complement participates in an activation loop with IFNγ and may contribute to GF. All patients were prospectively screened for TMA and terminal complement activation (plasma sC5b9). Patients who received eculizumab were dosed based on standard weight-based guidelines. Methylprednisolone was administered in 12 transplants at the time of emapalumab therapy (within 72 hours) for various indications, including immunomodulatory effects. Tocilizumab (n=1) and daratumumab (n=1) were also used for immunomodulatory effects in select transplants based on clinician discretion. G-CSF therapy (Granulocyte-Colony Stimulating Factor therapy, e.g., lenograstim, filgrastim, long-acting (pegylated) filgrastim, lipegfilgrastim)) was administered concurrently or started within 24 hours of the first dose of emapalumab in 72% ( 18/25) of transplants. G-CSF therapy was initiated a median of 5 days prior to emapalumab dosing (range, 33 days before first dose of emapalumab to 1 day after first dose of emapalumab) and patients who received GCSF prior to emapalumab did not show a response to G-CSF alone.

Patients treated at European centers (n=11) were deemed to be at high-risk for graft failure, as described in the methods section, and received emapalumab prophylactically with the aim of preventing graft failure. The median age at HSCT in this cohort was 2 years (range 0.6-17). Six out of 11 patients suffered an episode of graft failure in a prior HSCT. The remaining 5 patients were deemed high risk for graft failure in their first HSCT based on donor mismatch, underlying diagnosis and/or stem cell source/manipulation. Emapalumab was given before or immediately after the infusion of the graft and continued twice a week until sustained engraftment or graft failure. Of note, six patients with primary HLH received emapalumab maintenance therapy prior to transplant, as detailed in Table 2. The median emapalumab dose was 3 mg/kg (range 1-6) and the median number of doses from day −1 to completion was 8 (range 5-11). Two patients in the prophylaxis group received anakinra before HSCT for the treatment of hyperinflammation or refractory pleural/pericardial effusions and continued this therapy until engraftment.

### Safety

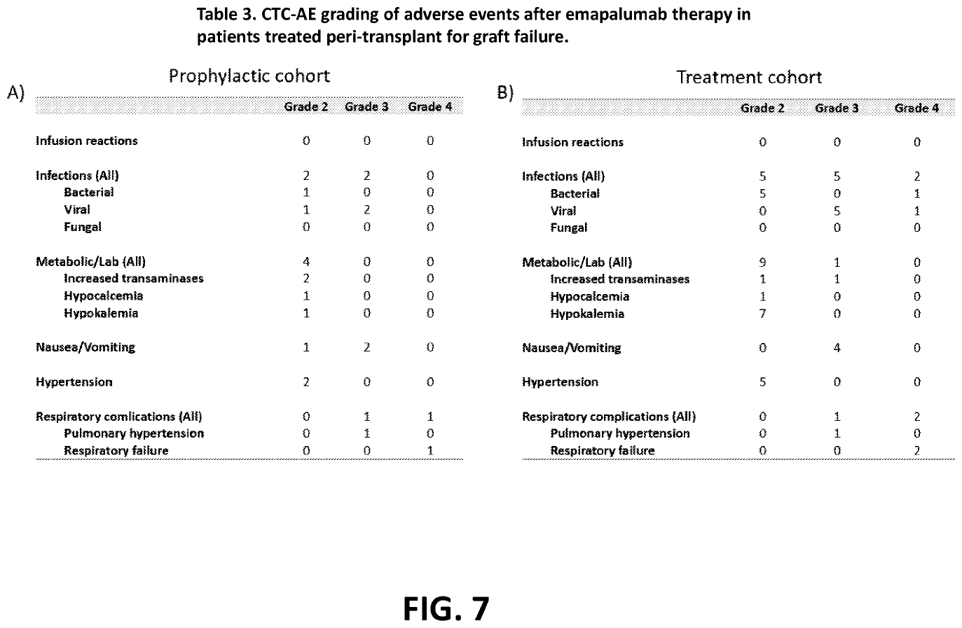

Emapalumab (alone or in combination with other monoclonal antibodies) was well tolerated with no infusion reactions reported. Adverse events recorded were in line with common toxicities related to HSCT in pediatric patients (see Table 3 for details); no treatment-related emergent adverse event was recorded. Also, infectious events did not differ with those occurring after standard transplants. No infection by mycobacteria, shigella, campylobacter and salmonella species, theoretically facilitated by inhibition of IFNγ (18), was recorded. Additionally, one patient (P9), who developed cutaneous Bacille Calmette Guerin infection during conventional treatment of his primary HLH, did not have recurrence during treatment with emapalumab, although the child was receiving antitubercular treatment, nor thereafter. No meningococcal infections occurred in patients who received eculizumab. FIG. 7. depicts Table 3: CTC-AE grading of adverse events after emapalumab therapy in patients treated peri-transplant for graft failure. All events were scored using CTC-AE v5 criteria. Infections were included if they occurred after the dose of emapalumab was administered. Infections that were already present at the time of emapalumab dosing were not included. No adverse events were directly attributable to emapalumab therapy. FIG. 7, panel A) depicts adverse events in 11 patients treated prophylactically with emapalumab. FIG. 7, panel A) depicts B) adverse events in 20 patients who received treatment emapalumab at Cincinnati Children's Hospital Medical Center for suspected graft failure.

### Efficacy

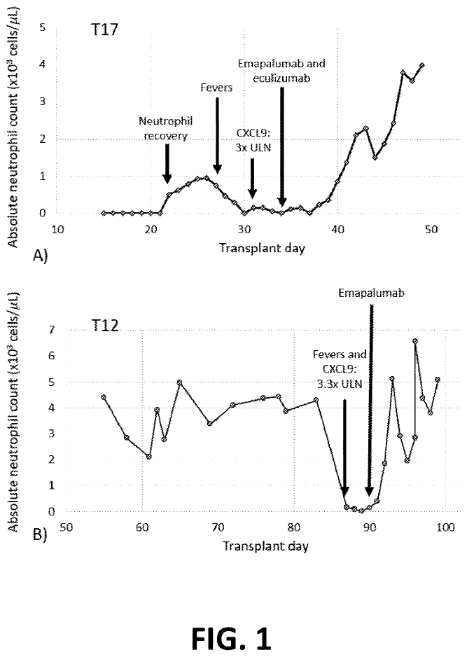

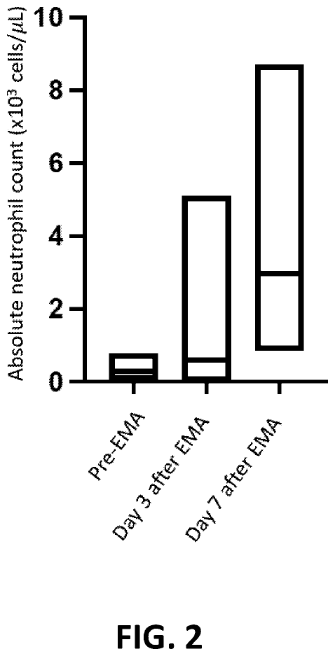

In the treatment group, 56% ( 14/25) of HSCTs had resolution of graft failure concerns after emapalumab therapy, while graft failure occurred in the remaining 44% ( 11/25) of HSCTs. Primary graft failure occurred in 2 transplants (both were patient T1), while the remaining 9 graft failures occurred after initial neutrophil engraftment and were therefore secondary graft failures. Representative ANC curves in patients with declining ANCs and concern for graft failure who responded to emapalumab therapy are shown in FIG. 1. Detailed patient demographics for these subjects are found in Table 2. FIG. 1, Panel A: Patient T17 is a 3-year-old male with dyskeratosis congenita who developed high fevers (Tmax 40.3 Celsius) that correlated with a sharp ANC decline after initial engraftment and elevated CXCL9. The patient was already receiving GCSF since day 1. Methylprednisolone was given on day 32 and 33 without immediate improvement. Emapalumab and eculizumab were then given on day 34 and stable improvement in counts followed. FIG. 1, Panel B: Patient T12 is 5-year-old female with macrophage activation syndrome who developed fevers (Tmax 38.9 Celsius) that correlated with a steep ANC decline and abrupt rise in CXCL9. The patient was started on GCSF on day 87 without immediate improvement in counts. Emapalumab and methylprednisolone were given on day 89 and her ANC improved rapidly. Patients who developed sudden neutropenia after initial neutrophil engraftment and responded to emapalumab therapy (n=6) had a prompt rise in their ANC within 3 days of emapalumab treatment (FIG. 2). Referring to FIG. 2, six patients achieved neutrophil engraftment after transplant, developed neutropenia and concern for secondary graft failure, and were successfully treated with emapalumab. The median absolute neutrophil count (ANC) for each timepoint is marked by the solid line and the median ANC values are: pre-emapalumab=0.29×10³ cells/μL, day 3 after emapalumab=0.6×10³ cells/μL, day 7 after emapalumab=2.97×10³ cells/μL. The box extends to the minimum and maximum value at each timepoint. All 6 patients had elevated CXCL9 levels prior to receiving emapalumab (patients T5, T6, T12, T13, T17 and T20 in Table 2). Of note, T20 had two separate and distinct events concerning for graft failure. This ANC trend is from the second event which occurred on day 97 (the first event occurred on day 6, prior to initial ANC engraftment). EMA=emapalumab.

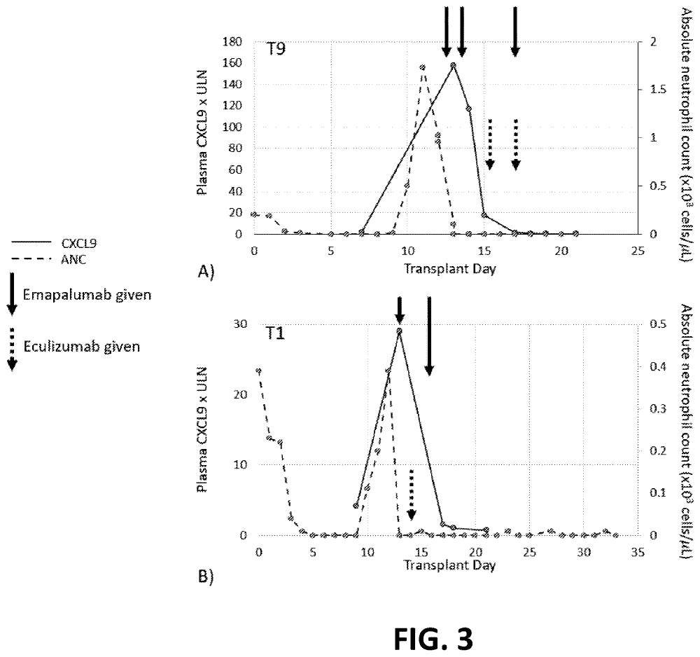

Multiple emapalumab doses were administered in seven out of the eleven HSCTs that resulted in graft failure in the treatment cohort, likely reflecting uncontrolled IFNγ activation. As described above, repeat dosing was based on real-time CXCL9 levels and the persistence of signs/symptoms of graft failure; therefore, patients who received multiple doses were more likely to have persistent signs of graft failure. CXCL9 kinetics are shown for 2 representative subjects who suffered graft failure despite emapalumab and eculizumab therapy (FIG. 3). Detailed patient demographics for the subjects in FIG. 3 are found in Table 2. FIG. 1, Panel A: Patient T9 is a 17-year-old male with severe aplastic anemia who developed high fevers shortly after ANC engraftment. This was associated with a massive CXCL9 elevation (157.8×ULN) and a drop in ANC to 0×10³ cells/μL despite concurrent GCSF therapy. Emapalumab (3 doses), eculizumab (Q24-Q72 hour dosing) and methylprednisolone were administered however the patient suffered graft failure. FIG. 1, Panel B: Patient T1 is a 17-year-old patient with CTLA4 deficiency who developed high fevers following his first HSCT. He showed early signs of neutrophil recovery but then developed worsening neutropenia and had an elevated CXCL9. He received emapalumab (2 doses) and eculizumab but still suffered primary graft failure. Overall survival (OS) in the treatment group was 67% ( 14/21). Causes of death included arrhythmia from sepsis and electrolyte derangements (n=1), posterior reversible encephalopathy syndrome with culture negative sepsis (PRES, n=1), pneumonitis (n=2), diffuse alveolar hemorrhage (DAH, n=2) and multiple viral infections (n=1).

In the prophylaxis group, 10 out of 11 patients engrafted at a median of 13.5 days (range 12-21); platelet recovery occurred at a median of 10 days (range 8-32). Full donor chimerism at day +28 was observed in all patients who engrafted. Five out of six patients with a prior history of graft failure engrafted and achieved full donor chimerism. Neither cases of secondary graft failure, nor cases of poor graft function were reported. With a median follow-up of 3.4 years for surviving patients, 9 out of 11 patients in the prophylaxis group are alive in remission of disease. Two patients died of transplant-related complications at 43 and 628 days after HSCT, respectively, with the projected 5-year overall survival being 79.5% (95% CI 39.3-94.5). Causes of death were acute GVHD and uncontrolled TMA, respectively.

### CXCL9 Levels in Peripheral Blood

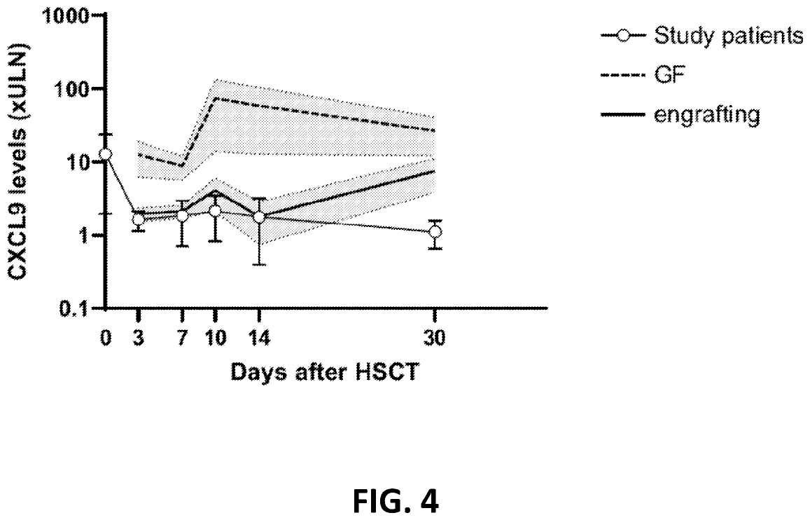

All patients in the treatment group at Cincinnati Children's Hospital Medical Center were monitored for CXCL9 levels in real-time and all patients treated at other US centers had CXCL9 levels center prior to emapalumab dosing. In total, 80% ( 20/25) of CXCL9 levels were elevated in the treatment cohort prior to emapalumab dosing. The median CXCL9 level prior to emapalumab dosing was elevated 2.4×ULN for the test (range, 0.3-157.8). The highest observed CXCL9 level prior to emapalumab dosing was elevated 157.8× the ULN and this patient suffered graft failure. The median pre-emapalumab CXCL9 level in patients who ultimately suffered graft failure in the treatment cohort was 3.3×ULN (range, 0.7-157.8) compared to a median of 2.2×ULN (range, 0.3-30.4) in subjects who had signs of graft failure but did not suffer graft failure (p=0.24). In the prophylaxis group, 6 patients underwent monitoring of CXCL9. Five of these patients engrafted and CXCL9 levels in engrafted patients resembled those we observed in a control group of patients we previously reported (FIG. 4). Referring to FIG. 4, CXCL9 data from 6 patients who had CXCL9 measurements and engrafted in the current study are shown. Individual points are shown as mean±standard error of mean (SEM). These data are in comparison to the CXCL9 mean±SEM of patients from our prior study (without emapalumab intervention) who experienced either graft failure (GF) or primary engraftment (engrafting). Patients having high-risk status treated prophylactically with emapalumab in the current study have a similar CXCL9 trend as patients who engrafted in the prior study and have lower CXCL9 levels than patients who experience graft failure in the prior study (Merli et al., Haematologica 2018).

### Discussion

In this study, potential uses of emapalumab for the prevention of graft failure using two different approaches were explored: prophylaxis or treatment. Multiple prior studies support an important contribution of INF-γ to the pathophysiology of immune-mediated graft failure and a limited number of case reports have described the use of emapalumab to prevent graft failure after HSCT. To Applicant's knowledge, the current study therefore contains the largest cohort of HSCTs (n=36) that used emapalumab therapy for this novel purpose.

The treatment cohort in this study combined post-transplant laboratory and clinical monitoring with pre-transplant graft failure risk factors to promptly identify patients with signs of graft failure who may benefit from emapalumab therapy. Emapalumab was well-tolerated in the treatment cohort and no infection was directly attributable to emapalumab. Nearly sixty percent of patients who received emapalumab had resolution of graft failure concerns, this finding suggesting that emapalumab may be an effective intervention to reverse impending graft failure. Elevated plasma CXCL9 levels were observed at the time of emapalumab treatment in most patients, which confirms the presence of systemic IFNγ pathway activation at the time of intervention. Five patients who received emapalumab in the treatment cohort did not have an elevated plasma CXCL9 level at the time of emapalumab therapy and two of these patients developed graft failure. This may reflect the timeliness of our intervention (e.g., IFNγ activation may have occurred later) and/or suggests multiple immunologic pathways are involved in mechanisms of graft failure after HSCT. Plasma CXCL9 levels were notably higher in patients who went on to develop graft failure in the current study, this suggested there may be a threshold of CXCL9 activation that, if crossed, leads to irreversible graft failure. Given these data and the known ability of emapalumab to remain in circulation for up to several weeks, one may therefore speculate that the earlier the intervention, the higher the probability of success.

Prophylactic emapalumab therapy is one potential approach to an early intervention strategy for patients identified as having a high-risk status for graft failure and this prophylactic approach was performed in the current study in two categories of patients: 1) patients with a prior history of graft failure; 2) patients with pre-transplant risk factors for graft failure but no prior history of graft failure. The prophylaxis cohort tolerated emapalumab without complication and there was no infection attributed to emapalumab treatment. Five out of six patients with a prior history of graft failure who received prophylactic emapalumab engrafted and achieved full donor chimerism. The second category of patients treated prophylactically had previously described risk factors for graft failure, but no prior history of graft failure. Patients tolerated therapy well, which is an important observation when considering the suitability of this treatment on a larger scale. Additionally, although the numbers are limited, these data compare favorably with our previous published results of ex vivo TCD haploidentical transplants in HLH, showing a risk of graft failure as high as 55% (Merli Blood Adv 2022).

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. All accessioned information (e.g., as identified by PUBMED, PUBCHEM, NCBI, UNIPROT, or EBI accession numbers) and publications in their entireties are incorporated into this disclosure by reference in order to more fully describe the state of the art as known to those skilled therein as of the date of this disclosure. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications may be made without departing from the spirit and scope of the invention. It is therefore intended to cover in the appended claims all such changes and modifications that are within the scope of this invention.

1. A method of treating an individual for transplant graft rejection comprising
a. identifying a risk level of the individual as either high-risk or moderate-risk, the identification comprising determining the presence of a risk factor selected from mismatched or haploidentical donor, ex vivo T-cell depleted graft, and history of prior graft failure; and
b. categorizing an individual with two identified risk factors as high-risk and an individual with one identified risk factor as moderate-risk.

2. The method of claim 1, further comprising administering an IFNγ neutralizing agent to the individual identified as high-risk.

3. The method of claim 1, further comprising
a. detecting in an individual classified as moderate-risk, the presence of a biomarker selected from fever, mixed chimerism, declining engraftment, an ANC decline of greater than or equal to 50% to absolute values less than 750, delayed engraftment, or CXCL9 greater than or equal to two times ULN or baseline value; and
b. administering the an IFNγ neutralizing agent to the individual when a the detected biomarker has a level that is elevated compared to a baseline level.

4. The method of claim 1, further comprising detecting a pre-conditioning baseline level of a second biomarker selected from CXCL9, sC5b-9, BAFF, CRP, sIL2r, and combinations thereof in the individual.

5. The method of claim 2, the administering comprising administering a second dose of emapalumab following fever, engraftment and/or ANC decline, CXCL9 level of greater than 2× upper limit of normal (ULN) or baseline.

6. The method of claim 2, wherein the IFNγ neutralizing agent is a human anti-IFNγ antibody.

7. The method of claim 2, wherein the IFNγ neutralizing agent is emapalumab.

8. The method of claim 7, the administering comprising administering 10 mg/kg of emapalumab to the individual.

9. The method of claim 7, the administering comprising administering 10 mg/kg of emapalumab to the individual at day 7 post-transplant.

10. The method of claim 7, the administering comprising administering 10 mg/kg of emapalumab to the individual at the time when the individual is categorized as high-risk.

11. A method for treating graft failure after allogeneic hematopoietic stem cell transplant (HSCT) in an individual in need thereof, comprising administering 1 to 10 mg/kg, as a 1-hour intravenous infusion of a human anti-IFNγ antibody, to the individual.

12. The method of claim 11, any wherein the treating is prophylactic.

13. The method of claim 11, wherein the individual is a pediatric individual.

14. The method of any claim 11, wherein the individual is an adult.

15. The method of any claim 11, further comprising administering eculizumab to the individual.

16. The method of any claim 11, further comprising administering one or both of tocilizumab and daratumumab to the individual.

17. The method of any preceding claim, further comprising
a. detecting, at a second timepoint, one or both of the risk factor or the biomarker; and
b. administering a second dose of the IFNγ neutralizing agent when the risk factor or biomarker is detected.

18. The method of claim 17 wherein the biomarker is CXCL9.

19. The method of claim 11, further comprising administering G-CSF therapy prior to emapalumab.

20. A device comprising a processor configured to receive data obtained in claim 1.

21. (canceled)

22. (canceled)

23. (canceled)

24. (canceled)
